SEQUENCE LISTING (13 sequences)

SEQ ID NO: 1 - protein, 541 aa, synthetic construct
MEWSWVFLFFLSVTTGVHSSNELHQVPSNCDCLNGGTCVSNKYFSNIHWCNCPKKFGGQHCEIDKSKTGGGGAGGGGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGGGGAGGGGHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDANGRRLTESYCETWRTEAPSATGQASSLLGGRLLGQSAASCHHAYIVLCIENSFMTASK

SEQ ID NO: 2 - protein, 768 aa, synthetic construct
EVQLQQSGPELVKTGASVKISCKASGYSFTSYYMHWVKQSHGKSLEWIGEINPYNGGASYNQKIKGRATFTVDTSSRTAYMQFNSLTSEDSAVYYCARSIYGHSVLDYWGQGTSVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSEEDCMFGNGKGYRGKRATTVTGTPCQDWAAQEPHRHSIFTPETNPRAGLEKNYCRNPDGDVGGPWCYTANPRKLYDYCDVPQCAADVVMTQTPLTLSVTIGQPASISCKSSQSLLDSDGKTYLNWLLQRPGQSPKRLIYLVSKLDSGVPDRFTGSGSGTDFTLKISRVEAEDLVYYCWQGTHFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 813 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 816 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 376 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADG

SEQ ID NO: 6 - protein, 483 aa, synthetic construct
MEWSWVFLFFLSVTTGVHSSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGSGSGSPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSSEEDCMFGNGKGYRGKRATTVTGTPCQDWAAQEPHRHSIFTPETNPRAGLEKNYCRNPDGDVGGPWCYTANPRKLYDYCDVPQCAA

SEQ ID NO: 7 - protein, 580 aa, synthetic construct
MEWSWVFLFFLSVTTGVHSSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGSGSGSPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDANGRRLTESYCETWRTEAPSATGQASSLLGGRLLGQSAASCHHAYIVLCIENSFMTASK

SEQ ID NO: 8 - protein, 580 aa, synthetic construct; identical to SEQ ID NO: 7

SEQ ID NO: 9 - protein, 764 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSEEDCMFGNGKGYRGKRATTVTGTPCQDWAAQEPHRHSIFTPETNPRAGLEKNYCRNPDGDVGGPWCYTANPRKLYDYCDVPQCADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 633 aa, synthetic construct
SNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGSGSGSPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSS

SEQ ID NO: 11 - protein, 908 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSVDHGFLVTRHSQTIDDPQCPSGTKILYHGYSLLYVQGNERAHGQDLGTAGSCLRKFSTMPFLFCNINNVCNFASRNDYSYWLSTPEPMPMSMAPITGENIRPFISRCAVCEAPAMVMAVHSQTIQIPPCPSGWSSLWIGYSFVMHTSAGAEGSGQALASPGSCLEEFRSAPFIECHGRGTCNYYANAYSFWLATIERSEMFKKPTPSTLKAGELRTHVSRCQVCMRRTDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 606 aa, synthetic construct
NELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKPLVQECMVHDCADGSGSGSPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSSVDHGFLVTRHSQTIDDPQCPSGTKILYHGYSLLYVQGNERAHGQDLGTAGSCLRKFSTMPFLFCNINNVCNFASRNDYSYWLSTPEPMPMSMAPITGENIRPFISRCAVCEAPAMVMAVHSQTIQIPPCPSGWSSLWIGYSFVMHTSAGAEGSGQALASPGSCLEEFRSAPFIECHGRGTCNYYANAYSFWLATIERSEMFKKPTPSTLKAGELRTHVSRCQVCMRRT

SEQ ID NO: 13 - protein, 866 aa, synthetic construct
EVQLQQSGPELVKTGASVKISCKASGYSFTSYYMHWVKQSHGKSLEWIGEINPYNGGASYNQKIKGRATFTVDTSSRTAYMQFNSLTSEDSAVYYCARSIYGHSVLDYWGQGTSVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDANGRRLTESYCETWRTEAPSATGQASSLLGGRLLGQSAASCHHAYIVLCIENSFMTASKDVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0179209 A1
(43) Pub. Date: Jun. 5, 2025

(54) ANTI-CANCER N-TERMINAL FC CONJUGATED IMMUNOTHERAPEUTICS

(71) Applicant: Immunofyx, Winnipeg (CA)

(72) Inventor: Mark WILLIAMS, Winnipeg (CA)

(73) Assignee: Immunofyx, Winnipeg, MB (CA)

(21) Appl. No.: 18/845,425

(22) PCT Filed: Mar. 9, 2023
(86) PCT No.: PCT/CA2023/050316

§ 371 Date: Sep. 9, 2024

Related U.S. Application Data
(60) Provisional application No. 63/318,225, filed on Mar. 9, 2022.

(51) Int. Cl. C07K 16/28; A61P 35/00; C07K 14/78; C12N 9/68

(52) U.S. Cl. CPC C07K 16/2896; A61P 35/00; C07K 14/78; C12N 9/6435; C12Y 304/21007; C07K 2319/30

(57) ABSTRACT

Compounds for the prevention and treatment of cancer have a first binding peptide from a urokinase-type plasminogen receptor (uPAR) antagonist operatively linked to an antibody peptide operatively linked to a second binding peptide from an endostatin or plasminogen derived peptide sequence.

### CROSS-REFERENCE TO RELATED APPLICATION

This application claims priority from U.S. provisional application No. 63/318,225 filed Mar. 9, 2022.

### FIELD OF THE INVENTION

The invention relates to compounds for the prevention and treatment of cancer.

### CROSS-REFERENCE TO RELATED DOCUMENTS

This application is filed concurrently with a sequence listing named SEQUENCE LISTING 56219-3003 encoded to meet the WIPO standards.

### BACKGROUND

Cancer is a leading cause of death worldwide, second only to cardiovascular disease. For the most part, while leukemias (blood born cancers) and melanoma (skin cancers) are well served by current treatments, unfortunately solid tumors have been less well served. As such, the rate of death from solid tumors are a major concern for oncologists and represent a major unmet medical need.

Two fundamental processes are important with respect to solid tumors, specifically angiogenesis and metastasis.

Angiogenesis is a process wherein new blood vessels are grown to deliver more nutrients, however this natural process can be co-opted in many solid tumor cancers to feed the growth of a tumor. As a result, it is a particularly important target for cancer treatment for solid tumors. However, under normal, non-pathological conditions, the body can use endogenous molecules to prevent uncontrolled angiogenesis such as endostatin and plasminogen.

Cancer metastasis is the major cause of cancer morbidity and mortality and may account for up to 90% of cancer deaths. Although cancer survival rate has been significantly improved over the years, the improvement is primarily due to early diagnosis and cancer growth inhibition. Limited progress has been made in the treatment of cancer metastasis. Current treatments for cancer metastasis are mainly chemotherapy and radiotherapy, though the new generation anti-cancer drugs (predominantly neutralizing antibodies for growth factors and small molecule kinase inhibitors) do have indirect effects on cancer metastasis in addition to their direct effects on cancer growth.

In particular, there is currently limited treatment options comprising medicaments and uses thereof that limit tumour angiogenesis and limit tumour metastasis of solid tumours.

### SUMMARY OF INVENTION

Compounds for the prevention and treatment of cancer have a first binding peptide from a urokinase-type plasminogen receptor (uPAR) antagonist operatively linked to the N-terminus of an Fc domain, with additional anti-angiogenic activity conferred by endostatin and/or plasminogen derived peptide sequences fused to the C-terminus of the same Fc fusion protein.

The IgG may be a fragment of an Fc fusion peptide, for example a human Fc fusion peptide. The Fc fusion peptide may be an IgG protein. The Fc fusion protein may be selected from the group consisting of an IgG1 isotype Fc peptide, an IgG2 isotype Fc peptide, an IgG3 isotype Fc peptide, an IgG4 isotype Fc peptide.

The first binding peptide may be an N terminal fragment from uPAR. The first binding peptide may be a heavy chain of the first binding peptide only. The first binding peptide may be selected from the group consisting of EGF-like domain G, an N-terminal portion of the ATF from a urokinase-type plasminogen receptor (uPAR) antagonist, antibody 2G10, antibody ATN-658, antibody 8B12. The first binding peptide may be a fragment of heavy and light sequences of antibodies that block uPAR signaling.

The second binding peptide may be may be selected from the group consisting of a fragment of an endostatin, an endostatin, a fragment of a plasminogen, a P125 mutation, a kringle domain of plasminogen, kringle domains 1 to 5 of plasminogen, kringle domain 5 from a plasminogen. The second binding peptide may be a fragment of an endostatin and a fragment of a plasminogen.

There may be a linker peptide between the antibody peptide and the first binding peptide. There may be a linker peptide between the antibody peptide and the second binding peptide.

The compound may be a peptide coded by a sequence selected from the group consisting of SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, and SEQ ID No. 14. The compound may have at least 70%, at least 75%, at least 80%, at least 85%, at least 90%, at least 95%, or at least 99% identical to the peptide coded by SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, and SEQ ID No. 13.

The compound may be selected from the group consisting of G-Fc-Endostatin (P125A), ATN658-Fc-K5, Herceptin-Fc-ATF, Avastin-Fc-ATF, Human IgG1 Fc-linker-ATF, ATF-Linker-hIgG4 Fc-Kringle domain K5, ATF-Linker-hIgG4 Fc-Endostatin (P125A), ATF-Linker-hIgG4 Fc-Endostatin (P125A), Herceptin-Fc-K5, ATF-Fc (IgG4)-linker-Kringle Domains 1-3, Herceptin-Arrestin, ATF-huIgG4 Fc-Arrestin and ATN658-Endo.

Also taught are compound substantially homologous to the above mentioned compounds.

Also taught are nucleotides encoding the compounds.

Also taught is Herceptin operatively linked to Arrestin.

The compounds exhibit may anti-cancer activity. Thus also taught are methods of treating cancer comprising administration of a composition comprising the compound of the invention, and the use of the compounds for treatment of cancer. The cancer may be a solid cancer. The methods may inhibit metastasis.

### BRIEF DESCRIPTION OF THE DRAWINGS

FIG. 1 provides a block diagram of the fusion of the uPAR inhibitor to the N-terminus of an IgG Fc domain and anti-angiogenic sequences derived from endostatin and/or plasminogen fused to the C-terminus of the Fc domain. Linkers are optionally added between the different domains.

FIG. 2 provides the trace chromatograms of G-Fc-Endostatin (P125A) under reducing and non-reducing conditions

FIG. 3A provides the effect of G-Fc-Endostatin (P125A) also called construct 1, compared with the anti-cancer agent Avastin (positive control) in the zebrafish model on tumor volume and metastases.

FIG. 3B provides the effect of G-Fc-Endostatin (P125A) on angiogenesis in a separate experiment.

FIG. 4 shows an SDS-PAGE analysis of ATN658-Fc-K5.

FIG. 5 shows an SDS-PAGE analysis of Herceptin-Fc-ATF.

FIG. 6 shows an SDS-PAGE analysis of Avastin-Fc-ATF.

FIG. 7 shows an SDS-PAGE analysis of Human IgG1 Fc-linker-ATF.

FIG. 8 shows an SDS-PAGE analysis of ATF-Linker-hIgG4 Fc-Kringle domain K5.

FIG. 9 shows an SDS-PAGE analysis of ATF-Linker-hIgG4 Fc-Endostatin (P125A).

FIG. 10 shows an SDS-PAGE analysis of ATF-Linker-hIgG4 Fc-Endostatin (P125A).

FIG. 11 shows an SDS-PAGE analysis of Herceptin-Fc-K5.

FIG. 12 shows an SDS-PAGE analysis of ATF-Fc (IgG4)-linker-Kringle Domains 1-3.

FIG. 13 shows an SDS-PAGE analysis of Herceptin-Arrestin.

FIG. 14 shows an SDS-PAGE analysis of ATF-huIgG4-Fc-Arrestin.

FIG. 15 shows an SDS-PAGE analysis of ATN658-Endo.

FIG. 16 is a graph showing normalized tumor size as effected by various Avastin and Herceptin concentrations and a graph showing number of metastases as effected by various Avastin and Herceptin concentrations.

FIG. 17 is a graph showing normalized tumor size as effected by various ATF constructs.

FIG. 18 is a graph showing normalized tumor size as effected by various Avastin and Herceptin concentrations and a graph showing normalized tumor size as effected by various constructs.

FIG. 19 is a graph showing normalized tumor size as effected by various constructs.

FIG. 20 is a graph showing normalized tumor size as effected by various constructs.

FIG. 21 is a graph showing number of metastases as effected by various constructs.

FIG. 22 is a graph showing normalized tumor volume reduction as effected by various constructs.

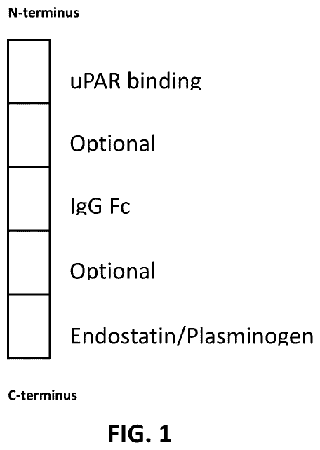

### DETAILED DESCRIPTION

The present invention relates to the combination of a urokinase-type plasminogen receptor (uPAR) antagonist with additional anti-angiogenic activity conferred by endostatin and/or plasminogen derived sequences in the same Fc fusion protein.

The present invention uses endostatin or plasminogen derived sequences in combination of uPA-uPAR interaction inhibitors, and proteins that target uPAR/vitronectin interactions in order to improve upon the anti-cancer activity of molecules simply inhibiting uPAR activity.

The present invention uses the kringle domains of plasminogen fused to the C-terminus of an antibody.

The proteins used in this invention are entirely endogenous sequences, thus there is little chance of unwanted immunogenicity against the therapeutics, which means the proteins should be able to be given repeatedly, even as an mRNA.

Accordingly, the present invention also provides DNA, RNA and mRNA based treatments. DNA may be administered, reach the damaged cells, enter the cell and express the proteins of the invention. Multiple delivery techniques can be used. For example, DNA may be entered into an engineered virus to deliver the DNA into a chromosome. Naked DNA approaches can also be used. Alternatively, one can administer a gene that causes a needed protein to be expressed.

In somatic cell gene therapy (SCGT), the therapeutic genes are transferred into any cell other than a gamete, germ cell, gametocyte, or undifferentiated stem cell. Therapeutic DNA (either integrated in the genome or as an external episome or plasmid) is used to treat disease.

In germline gene therapy (GGT), germ cells (sperm or egg cells) are modified by the introduction of functional genes into their genomes. Modifying a germ cell causes all the organism's cells to contain the modified gene. The change is therefore heritable and passed on to later generations.

In in vivo gene therapy, a vector (typically, a virus) is introduced to the patient, which then achieves the desired biological effect by passing the genetic material (e.g. for the therapeutic protein) into the patient's cells. In ex vivo gene therapies, such as CAR-T therapeutics, the patient's own cells (autologous) or healthy donor cells (allogeneic) are modified outside the body (hence, ex vivo) using a vector to express a particular protein, such as a chimeric antigen receptor.

The fusion protein constructs of the present invention have the advantage that they are a single chain, unlike antibodies with two chains (the heavy and light chain) which means only one mRNA that needs to be delivered.

The invention herein concerns the combining an Endostatin or Plasminogen derived sequences (or fragments thereof) with a uPAR binding domain with an intermediary Fc domain, resulting in a novel anti-cancer agent.

The N-terminal uPAR binding domain can be from sequences that block uPA and uPAR binding such a peptide ligand, the amino terminal fragment from uPA (or a fragment thereof,) or the sequences responsible of antibody 2G10. The uPAR binding domain can also be from heavy and light sequences of antibodies that block uPAR signaling such as those found in antibodies ATN-658 or 8B12.

A linker region is optionally added between the domains and the antibody Fc domain.

The isotype of the Fc domain (i.e. IgG1, 2, 3 or 4) can be selected pending a preference of improved half-life and/or ADCC and complement activity is desired. For example, in some situations, an IgG1 isotype may be preferred to increase the anti-cancer activity of the antibody by increasing ADCC. In other situations, an IgG4 isotype may be preferred for example to reduce ADCC which may allow for less toxicity and thereby permit higher doses. In some cases, an IgG3 Fc which is intermediate in ADCC activity to either IgG1 or IgG4 may be preferred. Note the Fc domain forms a natural homodimer.

The C-terminal anti-angiogenic domains are derived from endostatin (or a fragment thereof) and plasminogen kringle domain sequences, preferably the Kringle 5 domain. The use of combinations of sequences of endostatin and plasminogen together in the same molecule is also claimed.

Various protein based therapeutics have been utilized to block angiogenesis with known endogenous sequences of endostatin and plasminogen.

Endostatin is 20 kDa carboxyl-terminal fragment of type XVIII collagen that is present in walls and basement membranes of blood vessels and plays an important role in endothelial cell adhesion and cytoskeletal organization. Endogenous endostatin inhibits migration and induces apoptosis in endothelial cells, inhibits tumor growth, and impairs blood vessel maturation in wound healing. It is thought to interfere with the proangiogenic action of growth factors such as basic fibroblast growth factor and VEGF, and is known to inhibit at least 65 different tumor types. Additionally, a study measuring changes in gene expression in human dermal microvascular cells following treatment with endostatin demonstrated a downregulation of several proangiogenic pathways as well as the upregulation of many antiangiogenic genes.

Endostatin (trade name Endostar) is a protein that specifically inhibits endothelial proliferation and potently inhibits angiogenesis and tumor growth.

In 2005, a novel recombinant human endostatin, Endostar was approved by China's State Food and Drug Administration (SFDA) for the treatment of non-small cell line lung cancer. As such it has had a long history of human use, but surprisingly has never been approved in the US or Europe.

The half-life of the protein in the body is only 10 hours. As such Endostar is administered intravenously once per day over four hours over 14 days. This pharmacokinetic profile unavoidably causes inconvenience, affects quality of life and may reduce patient compliance.

A more active form of Endostar (P125A) has been identified. In addition, an N-terminal fragment of Endostatin was reported to possess a significant portion of the activity of Endostatin. With wild type Endostatin it has been reported the majority of the effects of endostatin can be ascribed to the N-terminal sequence (1-27) (Sjin RMTT, Satchi-Fainaro R, Birsner A E, Ramanujam V M S, Folkman J, Javaherian K. A 27-amino-acid synthetic peptide corresponding to the NH 2-terminal zinc-binding domain of endostatin is responsible for its antitumor activity. Cancer Res. 2005;65(9):3656-3663. doi:10.1158/0008-5472.CAN-04-1833).

Endostatin (P125A) has been fused to the C-terminus of other proteins such as Herceptin, an approved drug to block growth factor signals, which improved the activity of the original Herceptin (Shin S U, Cho H M, Merchan J, et al. Targeted delivery of an antibody-mutant human endostatin fusion protein results in enhanced antitumor efficacy. Mol Cancer Ther. 2011; 10(4):603-614. doi:10.1158/1535-7163.MCT-10-0804).

Plasminogen is an endogenous protein that can be cleaved by urokinase activated plasmin and contains five kringle (K) domains of ˜80 residues each. Endogenous angiostatin is a 38 kDa amino-terminal fragment of plasminogen, and studies using recombinant angiostatin demonstrated tumor inhibitory activity resides in domains K1-3⁸. Angiostatin was originally isolated from tumor bearing mice, ⁶ and has both potent antiangiogenic activity and antiproliferative activity toward endothelial and cancer cells.⁷ Larger fragments of plasminogen that contain all 5 kringle domains (K1-5) may be even more active than angiostatin alone (K1-3) (Cao R, Wu HL, Veitonmäki N, et al. Suppression of angiogenesis and tumor growth by the inhibitor K1-5 generated by plasmin-mediated proteolysis. Proc Natl Acad Sci U S A. 1999;96(10):5728-5733. doi:10.1073/pnas.96.10.5728).

Recent evidence supports dual antitumor mechanisms for plasminogen derivatives, one affecting angiogenesis and another targeting tumor cells directly. (Sun Q, Xu Q, Dong X, et al. A hybrid protein comprising ATF domain of pro-UK and VAS, an angiogenesis inhibitor, is a potent candidate for targeted cancer therapy. Int J cancer. 2008;123(4):942-950. doi:10.1002/IJC.23537).

Kringle 5 (K5), like angiostatin, is a byproduct of the proteolytic cleavage of plasminogen. In a recent study, Ansell et al., demonstrated K5 functions as a competitive antagonist of hepatocyte growth factor (HGF). (Cho H M, Rosenblatt J D, Kang Y S, et al. Enhanced inhibition of murine tumor and human breast tumor xenografts using targeted delivery of an antibody-endostatin fusion protein. Mol Cancer Ther. 2005;4(6):956-967. doi:10.1158/1535-7163.MCT-04-0321).

HGF contains kringle motifs and promotes angiogenesis by stimulating the tyrosine kinase receptor Met. In addition to its potent angiostatic role, K5 can direct a potent antitumor response with its ability to recruit tumor-associated neutrophils and Natural Killer T cells.

Thus plasminogen has multiple kringle domains which appear to be active alone (angiostatin (K1-4) and K5) and in combination. Thus use of plasminogen derived sequences, in particular the K5 domain with other anti-cancer proteins has been reported (Wang H, Yang Z, Gu J. Therapeutic targeting of angiogenesis with a recombinant CTT peptide-endostatin mimic-kringle 5 protein. Mol Cancer Ther. 2014;13(11):2674-2687. doi:10.1158/1535-7163.MCT-14-0266).

One of the major events that underlie metastasis is the proteolytic degradation of the extracellular matrix (ECM) to promote tumor cell invasion, migration, and homing to distant organs. Even though several protease systems are implicated in this process, a large body of evidence identified the urokinase-type plasminogen activator receptor (uPAR) system as a central player in mediating proteolysis during cancer invasion and metastasis. The uPA-mediated degradation of the ECM is also crucial for the initiation of angiogenesis.

uPAR is a is a GPI-anchored cell membrane receptor that localizes urokinase (uPA) proteolytic activity on the cell surface. Its main function is focusing of urokinase (uPA) proteolytic activity, responsible for degradation of ECM components, on the cell surface. uPAR expression is increased in many human cancers and correlates with a poor prognosis and early invasion and metastasis and has been reported on as many as 80% of tumors. At some point, the uPA-UPAR complex is often internalized to break apart the complex, and unoccupied UPAR may be recycled to the surface.

There are two broad mechanisms to inhibit the function of UPAR; (1) binding of uPA with uPAR, and (2) preventing the cell signaling that can occur by UPAR which involves both uPA binding and its subsequent binding with co-receptors such as integrins.

The binding of uPA with uPAR is instrumental for the activation of plasminogen to plasmin, which in turn initiates a series of proteolytic cascade to degrade the components of the extracellular matrix, and thereby cause tumor cell migration from the primary site of origin to a distant secondary organ.

uPA is often separated into two regions. The first region which is responsible for binding of uPA to UPAR is the amino terminal fragment (ATF). The ATF can be further divided into two domains: an epidermal growth factor (EGF)-like domain and a kringle domain. The “G” domain is the EGF-like region and the “K” is the kringle domain. The majority of the binding occurs with just the EGF-like domain alone, but the entire ATF region including both the EGF-like domain and the kringle domain does have even greater binding the just the EGF-like domain alone. In addition, short peptide sequences unrelated to the native ATF sequence have been identified which also compete with uPA binding to UPAR (Goodson R J, Doyle M V., Kaufman S E, Rosenberg S. High-affinity urokinase receptor antagonists identified with bacteriophage peptide display. Proc Natl Acad Sci U S A. 1994;91(15):7129-7133. doi:10.1073/pnas.91.15.7129). Indeed an ATF-Fc fusion (Chinese patent No. CN101050237) has been shown to reduce tumor growth and metastases (Hu X W, Duan H F, Gao L H, et al. Inhibition of tumor growth and metastasis by ATF-Fc, an engineered antibody targeting urokinase receptor. Cancer Biol Ther. 2008;7(5):651-659. doi:10.4161/cbt.7.5.5643).

An antibody (2G10) was identified from a phage display library that prevented uPA association with uPAR, been reported to prevent uPAR and uPA binding (Duriseti S, Goetz D H, Hostetter D R, LeBeau A M, Wei Y, Craik C S. Antagonistic anti-urokinase plasminogen activator receptor (uPAR) antibodies significantly inhibit uPAR-mediated cellular signaling and migration. J Biol Chem. 2010;285(35):26878-26888. doi:10.1074/jbc.M109.077677). Non natural smaller peptides which inhibit the uPA/uPAR interaction have been identified from combinatorial libraries as well.

The second region contains the enzymatic activity of uPA. It is initially an inactive pro-enzyme which when activated, enzymatically cleaves plasminogen and activates that enzyme to plasmin which mediates degradation of the extracellular matrix.

Once uPA binds to UPAR, the complex of the two proteins is also able to interact with other proteins (co-receptors) at the surface of the cancer cell such as integrins. When UPAR interacts with these coreceptors, mostly through Domain 3 of its three domains, this results in activation of cell signalling pathways that hasten the cancer progression including metastases.

Targeting the signaling aspect of UPAR function, unlike preventing the binding of uPA to UPAR where the therapeutic protein “competes” for binding with uPA for UPAR receptor occupancy, is “non-competitive” with uPA binding to UPAR and may be more effective at preventing metastases by blocking the cell signaling events that drive the cancer cell to metastasize. An example of this would be the binding sequences (CDRs) of the antibody ATN-658 (Mahmood N, Arakelian A, Khan H A, Tanvir I, Mazar A P, Rabbani S A. uPAR antibody (huATN-658) and Zometa reduce breast cancer growth and skeletal lesions. Bone Res. 2020;8(1). doi:10.1038/S41413-020-0094-3). Another antibody (8B12) has been reported against conformation specific uPAR to reduce uPA binding to vitronectin and can reduce tumor volume (Yuan C, Guo Z, Yu S, Jiang L, Huang M. Development of inhibitors for uPAR: blocking the interaction of uPAR with its partners. Drug Discov Today. 2021;26(4):1076-1085. doi:10.1016/j.drudis.2021.01.016)

There are reports of combining inhibitors of uPA-uPAR (e.g. ATF) with inhibitors such as protease inhibitor domains as well as the anti-angiogenic functional domains of vasostatin. Similarly, there have been reports of Endostatin (P125A) fused to the C-terminus of other antibodies such as Herceptin.

The methods and techniques of the present disclosure are generally performed according to conventional methods well known in the art and as described in various general and more specific references that are cited and discussed throughout the present specification unless otherwise indicated.

The terms “polypeptide”, “peptide”, and “protein” are typically used interchangeably herein to refer to a polymer of amino acid residues. Amino acids may be referred to herein by either their commonly known three letter symbols or by the one-letter symbols recommended by the IUPAC-IUB Biochemical Nomenclature Commission. Each protein or polypeptide will have a unique function. The invention includes polypeptides and functional fragments thereof, as well as mutants and variants having the same biological function or activity.

In some embodiments, polymeric molecules (e.g., a polypeptide sequence or nucleic acid sequence) are considered to be “homologous” to one another if their sequences are at least 70%, at least 75%, at least 80%, at least 85%, at least 90%, at least 95%, or at least 99% identical.

In some embodiments a fragment of a nucleic acid sequence is a fragment of an open reading frame sequence. In some embodiments, such a fragment encodes a polypeptide fragment (as defined herein) of the protein encoded by the open reading frame nucleotide sequence.

The term “nucleic acid fragment” as used herein refers to a nucleic acid sequence that has a deletion. In some embodiments a fragment of a nucleic acid sequence is a fragment of an open reading frame sequence. In some embodiments, such a fragment encodes a polypeptide fragment (as defined herein) of the protein encoded by the open reading frame nucleotide sequence.

The term “construct” refers to a nucleic acid sequence encoding a protein, operably operatively linked to a promoter and/or other regulatory sequences.

The term “genomic sequence” refers to a sequence having non-contiguous open reading frames, where introns interrupt the protein coding regions.

As used herein, the terms “encoding”, “coding”, or “encoded” when used in the context of a specified nucleic acid mean that the nucleic acid may be the requisite information to guide translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

The term “percent sequence identity” or “identical” in the context of nucleic acid sequences refers to the residues in the two sequences which are the same when aligned for maximum correspondence. For instance, polynucleotide sequences can be compared using the computer program, BLAST (Altschul et al., J. Mol. Biol. 215:403-410 (1990); Gish and States, Nature Genet. 3:266-272 (1993).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 70%, 80%, 85%, or at least about 90%, or at least about 95%, 96%, 97%, 98% or 99% of the nucleotide bases, as measured by any well-known algorithm of sequence identity, such as BLAST, as discussed above.

As used herein, “heterologous nucleic acid sequence” is any sequence placed at a location in the genome where it does not normally occur. In some embodiments, the heterologous nucleic acid sequence is a natural phage sequence, albeit from a different phage.

A particular nucleic acid sequence also encompasses conservatively modified variants thereof (such as degenerate codon substitutions) and complementary sequences, as well as the sequence explicitly indicated. Thus, a nucleic acid sequence encoding a protein sequence disclosed herein also encompasses modified variants thereof as described herein. Substantially similar nucleic acid fragments of the instant invention may also be characterized by the percent identity of the amino acid sequences that they encode to the amino acid sequences disclosed herein, as determined by algorithms commonly employed by those skilled in this art.

“Operatively linked” or “operably linked” expression control sequences refers to a linkage in which the expression control sequence is contiguous with coding sequences of interest to control expression of the coding sequences of interest, as well as expression control sequences that act in trans or at a distance to control expression of the coding sequence.

A “coding sequence” or “open reading frame” is a sequence of nucleotides that encodes a polypeptide or protein. The termini of the coding sequence are a start codon and a stop codon. The disclosure also includes native, isolated, or recombinant nucleic acid sequences encoding a protein, as well as vectors and/or (host) cells containing the coding sequences for the protein.

The terms “Fc domain”, “Fc peptide” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, MD, 1991. A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

Fused or linked means that the components (e.g. a Fab molecule and an Fc domain subunit) are linked by peptide bonds, either directly or via one or more peptide linkers.

Fragments and variants of the disclosed nucleotide sequences and proteins encoded thereby are also encompassed by the present invention. By “fragment” a portion of the nucleotide sequence or a portion of the amino acid sequence and hence protein encoded thereby is intended. Fragments of a nucleotide sequence may encode protein fragments that retain the biological activity of the native protein. Accordingly, the present disclosure relates to any nucleic acid fragment comprising a nucleotide sequence that encodes all or a substantial portion of the amino acid sequences encoded thereby.

All publications and patents mentioned in this specification are herein incorporated by reference to the same extent as if each individual publication or patent was specifically and individually indicated to be incorporated by reference.

The foregoing description and certain representative embodiments and details of the invention have been presented for purposes of illustration and description of the invention. It is not intended to be exhaustive or to limit the invention to the precise forms disclosed. It will be apparent to practitioners skilled in this art that modifications and variations may be made therein without departing from the scope of the invention.

### EXAMPLES

### Example 1—Constructing G-Fc-Endostatin (P125A)

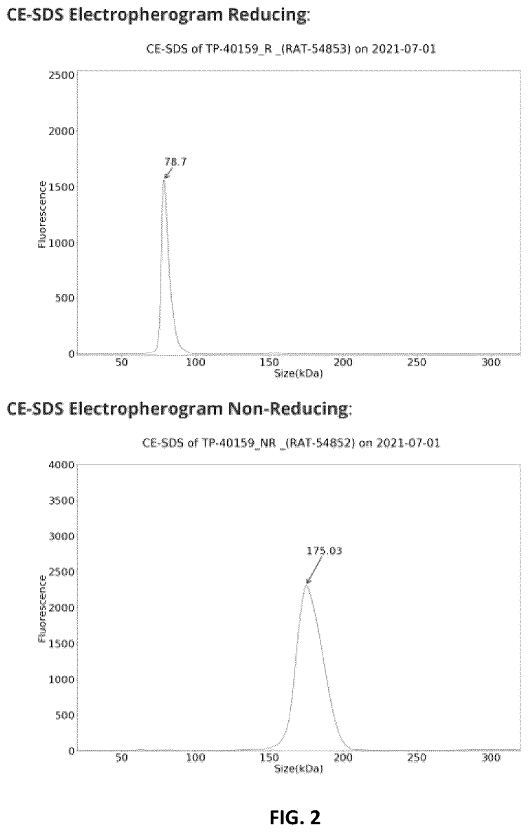

This construct following a signal export peptide uses the N-terminal portion of the ATF from uPA (UniProt P00749), specifically the EGF-like domain “G” (residues 21-69, in bold) fused via a linker (GGGGAGGGG) to a human Fc (of the IgG3 isotype) and then further linked (GGGAGGGG) to the endostatin protein (UniProt P39060, residues 1572-1754, with a P125A mutation in italics). The molecule was produced using standard molecular biology techniques and expressed using mammalian cell culture to produce pure target molecule following protein A purification (FIG. 2). The sequence is set out in SEQ ID No. 1, below.

MEWSWVFLFFLSVTTGVHSSNELHQVPSNCDCLNGGTCVSNKYFSNIHW
CNCPKKFGGQHCEIDKSKTGGGGAGGGGDTTHTCPRCPEPKSCDTPPPC
PRCPEPKSCDTPPPCPRCPEPKSCDTPPPCPRCPAPELLGGPSVFLFPP
KPKDTLMISRTPEVTCVVVDVSHEDPEVQFKWYVDGVEVHNAKTKPREE
QYNSTFRVVSVLTVLHQDWLNGKEYKCKVSNKALPAPIEKTISKTKGQP
REPQVYTLPPSREEMTKNQVSLTCLVKGFYPSDIAVEWESSGQPENNYN
TTPPMLDSDGSFFLYSKLTVDKSRWQQGNIFSCSVMHEALHNRFTQKSL
SLSPGKGGGGAGGGGHSHRDFQPVLHLVALNSPLSGGMRGIRGADFQCF
QQARAVGLAGTFRAFLSSRLQDLYSIVRRADRAAVPIVNLKDELLFPSW
EALFSGSEGPLKPGARIFSFDGKDVLRHPTWPQKSVWHGSDANGRRLTE
SYCETWRTEAPSATGQASSLLGGRLLGQSAASCHHAYIVLCIENSFMTA
SK

Signal Peptide
Gene of Interest; [symbol] /Endostatin

[symbol]

Tag (Hinge/Fc)

[symbol]

Protein: uPA (21-69)-Fc-Endostatin (P125A)
Gene ID/Reference:
UniProt P00749, residues 21-69
UniProt P33060, residues 1572-1754, mutation P125A
Isotype: Human IgG3 Fc

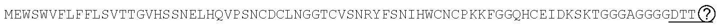

[symbol]

[symbol]

[symbol]

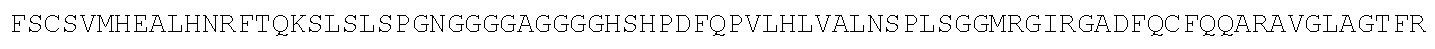

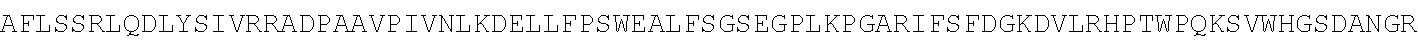

RLTESYCETWPTEAPSATGQASSLLGGPLLGQSAASCHHAYIVLCIENSFMTASK*

[symbol]

indicates data missing or illegible when filed

Cloning, expression and purification was carried out by methods known in the art.

FIG. 2 shows the trace chromatograms of G-Fc-Endostatin (P125A) under reducing and non-reducing conditions. Under reducing conditions interactions between two polypeptides is disrupted. However, under non reducing conditions, the interactions are preserved. Thus, two conditions allow us to identify protein-protein interactions. The single peak under both conditions indicates a stable, single compound.

### Example 2—In Vivo Anti-Cancer Testing Using Zebrafish Model

The zebrafish model is a remarkable short but predictive model to screen anti-cancer agents that has gained wide acceptance as a useful in vivo model.

### Zebrafish Tumor Xenograft (ZTX) Model

Transgenic Tg(fli1:EGFP)y1 zebrafish embryos were raised at 28° C. for 48 hours in E3 embryo medium containing 0.2 mM 1-Phenyl-2-Thiourea a.k.a. PTU. Unfertilized eggs or larvae that did not appear healthy or exhibited any obvious developmental defects were excluded before treatment onset.

PC3 cancer cells were labeled with the Dil red fluorescent dye. Approximately 700 (efficacy study) or 2500 (angiogenesis study) Dil-labeled PC3 cancer cells (red) were subcutaneously implanted into the perivitelline space of 2 days old larvae±test article at a given concentration.

Larvae in which tumor cells had been inadvertently injected into circulation or larvae with erroneous implantation of the tumor in the yolk rather than the perivitelline space were excluded from the study. Pictures of primary tumors were taken right after injection. Tumor-bearing embryos were incubated in E3/PTU medium for 72 hours at 36° C. After incubation, pictures of the primary tumors and the CVP were taken by using a red fluorescent filter. Larvae that died or were lost by other means during the study were excluded from the final analysis. Embryos that survived after 3 days of treatment were mounted and images of primary tumors and intratumoral blood vessels were taken using an upright Zeiss confocal LSM700.

### Analysis of Tumor Growth Inhibition and Metastasis

Images obtained right after implantation (day 0) and after 72 hours incubation (day 3) were analyzed by using in-house developed software. Tumor growth regression was calculated and normalized to the respective control. Metastasis was evaluated in the software and counted manually.

### Analysis of Tumor Angiogenesis

Images obtained of primary tumors in the red and green channel were analyzed using ImageJ v1.53f51. Primary tumor and intratumoral blood vessels area were measure. Tumor angiogenesis was calculated by normalizing the blood vessel area to the respective primary tumor area.

Avastin (bevacizumab) is a medication used to treat a number of types of cancers. It is a monoclonal antibody that functions as an angiogenesis inhibitor. It was approved for medical use in the United States in 2004.

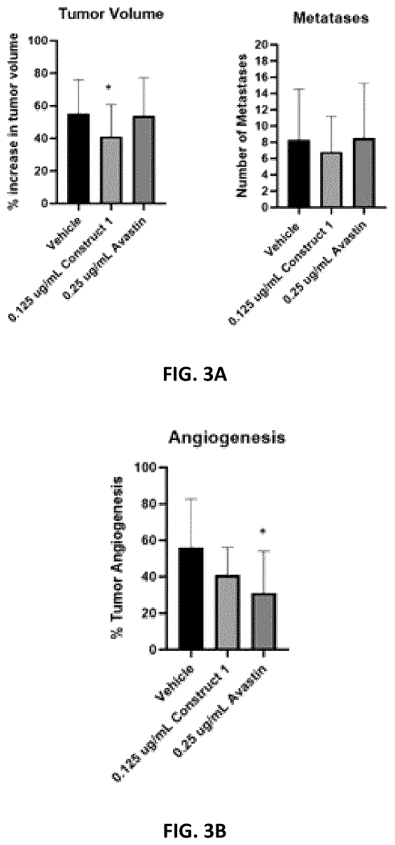

FIG. 3A provides the effect of G-Fc-Endostatin (P125A) also called construct 1, compared with the anti-cancer agent Avastin (positive control) in the zebrafish model on tumor volume and metastases. Construct 1 was shown to be a better inhibitor of tumor volume and metastases as compared to the vehicle and Avastin.

FIG. 3B provides the effect of G-Fc-Endostatin (P125A) on angiogenesis in a separate experiment. Construct 1 was shown to be a better inhibitor of Angiogenisis as compared to the vehicle and Avastin.

The significance (*p<0.05) was determined using two-tailed Student's t-test.

### Example 3—Constructing ATN658-Fc-K5

This fusion protein uses the heavy chain ATF658 bound to a linker and then to K5. The molecule was produced using standard molecular biology techniques and expressed using mammalian cell culture. The expression vectors of the fusion protein were transiently transfected and expressed in mammalian cells with chemically defined culture media. This protein was purified by Protein A affinity chromatography, dialysed with PBS, concentrated (if needed), and then subjected to 0.2 micron sterile filtration to get the bulk with high purity. The sequence is set out in SEQ ID No. 2, below.

Heavy chain (ATN658)-linker-K5
EVQLQQSGPELVKTGASVKISCKASGYSFTSYYMHWVKQSHGKSLEWIG
EINPYNGGASYNQKIKGRATFTVDTSSRTAYMQFNSLTSEDSAVYYCAR
SIYGHSVLDYWGQGTSVSVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQK
SLSLSPGKGGGGSGGGGSGGGGSSEEDCMFGNGKGYRGKRATTVTGTPC
QDWAAQEPHRHSIFTPETNPRAGLEKNYCRNPDGDVGGPWCYTANPRKL
YDYCDVPQCAA
Light chain (ATN658)
DVVMTQTPLTLSVTIGQPASISCKSSQSLLDSDGKTYLNWLLQRPGQSP
KRLIYLVSKLDSGVPDRFTGSGSGTDFTLKISRVEAEDLVYYCWQGTHF
PLTFGAGTKLELKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

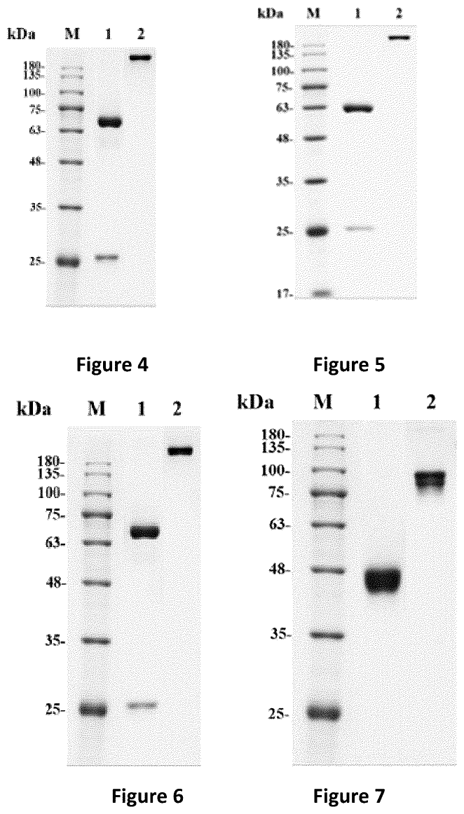

Purity was 99.6835% as determined by SEC-HPLC. Endotoxin: <1 EU/mg. Concentration was 1.2 mg/mL. Formulation Buffer was PBS, PH 7.4. As shown in FIG. 4, SDS-PAGE analysis of ATN658-Fc-K5 in β-mercaptoethanol-reduced (Lane 1) and non-reduced (Lane 2) conditions. Gel stained for 30 minutes with Coomassie Blue. As a result of different reduced proteins (Heavy chain and Light chain) migrate as about 63 kDa and 25 kDa, respectively. Non-reduced protein migrates about 180 kDa.

### Example 4—Constructing Herceptin-Fc-ATF

This fusion protein uses the heavy chain Herceptin bound to an Fc linker and then to ATF. The molecule was produced using standard molecular biology techniques and expressed using mammalian cell culture. The expression vectors of the fusion protein were transiently transfected and expressed in mammalian cells with chemically defined culture media. This protein was purified by Protein A affinity chromatography, dialysed with PBS, concentrated (if needed), and then subjected to 0.2 micron sterile filtration to get the bulk with high purity. The sequence is set out in SEQ ID No. 3, below.

Heavy chain (Herceptin)-linker-ATF
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGKGGGGSGGGGSGGGGSSNELHQVPSNCDCLNGGTCVSNKYF
SNIHWCNCPKKFGGQHCEIDKSKTCYEGNGHFYRGKASTDTMGRPCLPW
NSATVLQQTYHAHRSDALQLGLGKHNYCRNPDNRRRPWCYVQVGLKPLV
QECMVHDCADG
Light chain (Herceptin)
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Purity was 99.6621% as determined by SEC-HPLC. Endotoxin: <1 EU/mg. Concentration was 2 mg/mL. Formulation Buffer was PBS, PH 7.4.

As shown in FIG. 5, SDS-PAGE analysis of the construct in β-mercaptoethanol-reduced (Lane 1) and non-reduced (Lane 2) conditions. Gel stained for 30 minutes with Coomassie Blue. As a result of different reduced proteins (Heavy chain and Light chain) migrate as about 63-75 kDa and 25 kDa, respectively. Non-reduced protein migrates about 180 kDa.

### Example 5—Constructing Avastin-Fc-ATF

This fusion protein uses the heavy chain Avastin bound to an Fc linker and then to ATF. The molecule was produced using standard molecular biology techniques and expressed using mammalian cell culture. The expression vectors of the fusion protein were transiently transfected and expressed in mammalian cells with chemically defined culture media. This protein was purified by Protein A affinity chromatography, dialysed with PBS, concentrated (if needed), and then subjected to 0.2 micron sterile filtration to get the bulk with high purity. The sequence is set out in SEQ ID No. 4, below.

Heavy chain (Avastin)-linker-ATF
EVQLVESGGGLVQPGGSLRLSCAASGYTFTNYGMNWVRQAPGKGLEWVG
WINTYTGEPTYAADFKRRFTFSLDTSKSTAYLQMNSLRAEDTAVYYCAK
YPHYYGSSHWYFDVWGQGTLVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNH
YTQKSLSLSPGKGGGGSGGGGSGGGGSSNELHQVPSNCDCLNGGTCVSN
KYFSNIHWCNCPKKFGGQHCEIDKSKTCYEGNGHFYRGKASTDTMGRPC
LPWNSATVLQQTYHAHRSDALQLGLGKHNYCRNPDNRRRPWCYVQVGLK
PLVQECMVHDCADG
Light chain (Avastin)
DIQMTQSPSSLSASVGDRVTITCSASQDISNYLNWYQQKPGKAPKVLIY
FTSSLHSGVPSRFSGSGSGTDFTLTISSLQPEDFATYYCQQYSTVPWTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Purity was 98.2435% as determined by SEC-HPLC. Endotoxin: <1 EU/mg. Concentration was 2 mg/mL. Formulation Buffer was PBS, PH 7.4.

As shown in FIG. 6, SDS-PAGE analysis of the construct in β-mercaptoethanol-reduced (Lane 1) and non-reduced (Lane 2) conditions. Gel stained for 30 minutes with Coomassie Blue. As a result of different reduced proteins (Heavy chain and Light chain) migrate as about 63-75 kDa and 25 kDa, respectively. Non-reduced protein migrates about 180 kDa.

### Example 6—Constructing Human IgG1 Fc-Linker-ATF

This fusion protein uses Human IgG1 Fc-linker-ATF bound to. The molecule was produced using standard molecular biology techniques and expressed using mammalian cell culture. The expression vectors of the fusion protein were transiently transfected and expressed in mammalian cells with chemically defined culture media. This protein was purified by Protein A affinity chromatography, dialysed with PBS, concentrated (if needed), and then subjected to 0.2 micron sterile filtration to get the bulk with high purity. The sequence is set out in SEQ ID No. 5, below.

Human IgG1 Fc-linker-ATF
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSR
WQQGNVFSCSVMHEALHNHYTQKSLSLSPGKGGGGSGGGGSGGGGSSNE
LHQVPSNCDCLNGGTCVSNKYFSNIHWCNCPKKFGGQHCEIDKSKTCYE
GNGHFYRGKASTDTMGRPCLPWNSATVLQQTYHAHRSDALQLGLGKHNY
CRNPDNRRRPWCYVQVGLKPLVQECMVHDCADG

Purity was 97.8344% as determined by SEC-HPLC. Endotoxin: <1 EU/mg. Concentration was 2 mg/mL. Formulation Buffer was PBS, PH 7.4.

As shown in FIG. 7, SDS-PAGE analysis of the construct in β-mercaptoethanol-reduced (Lane 1) and non-reduced (Lane 2) conditions. Gel stained for 30 minutes with Coomassie Blue. As a result of different reduced proteins migrate as about 63-48 kDa, respectively. Non-reduced protein migrates about 75-100 kDa.

### Example 7—Constructing ATF-Linker-hIgG4 Fc-Kringle Domain K5

This fusion protein uses ATF bound to a linker then to Fc then to Kringle domain K5. The molecule was produced using standard molecular biology techniques and expressed using mammalian cell culture. The expression vectors of the fusion protein were transiently transfected and expressed in mammalian cells with chemically defined culture media. This protein was purified by Protein A affinity chromatography, dialysed with PBS, concentrated (if needed), and then subjected to 0.2 micron sterile filtration to get the bulk with high purity. The sequence is set out in SEQ ID No. 6, below.

ATF-Linker-hIgG4 Fc-Kringle domain K5
MEWSWVFLFFLSVTTGVHSSNELHQVPSNCDCLNGGTCVSNKYFSNIHW
CNCPKKFGGQHCEIDKSKTCYEGNGHFYRGKASTDTMGRPCLPWNSATV
LQQTYHAHRSDALQLGLGKHNYCRNPDNRRRPWCYVQVGLKPLVQECMV
HDCADGSGSGSPPCPPCPAPEFLGGPSVFLFPPKPKDTLMISRTPEVTC
VVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYS
RLTVDKSRWQEGNVFSCSVMHEALHNHYTQKSLSLSLGKGGGGSGGGGS
GGGGSSEEDCMFGNGKGYRGKRATTVTGTPCQDWAAQEPHRHSIFTPET
NPRAGLEKNYCRNPDGDVGGPWCYTANPRKLYDYCDVPQCAA

Purity was 98.6250% as determined by SEC-HPLC. Endotoxin: <1 EU/mg. Concentration was 2 mg/mL. Formulation Buffer was PBS, PH 7.4.

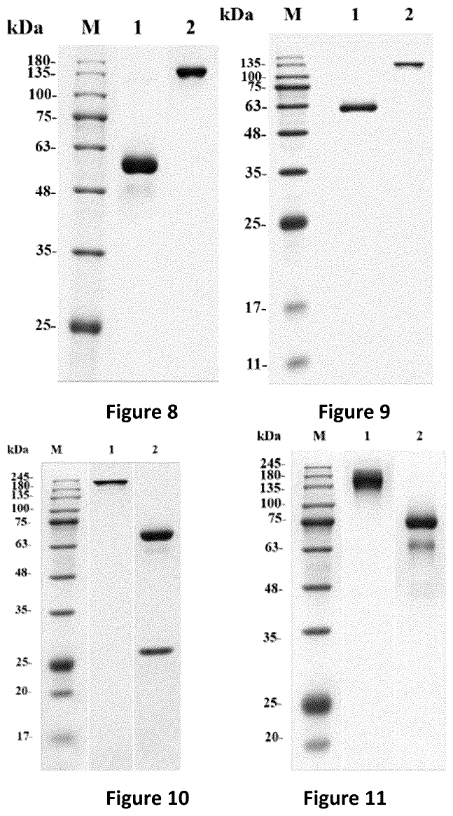

As shown in FIG. 8, SDS-PAGE analysis of the construct in β-mercaptoethanol-reduced (Lane 1) and non-reduced (Lane 2) conditions. Gel stained for 30 minutes with Coomassie Blue. As a result of different reduced proteins migrate as about 48-63 kDa. Non-reduced protein migrates about 135-180 kDa.

### Example 8—Constructing ATF-Linker-hIgG4 Fc-Endostatin (P125A)

This fusion protein uses the heavy chain ATF bound to a linker then to Fc and then to Endostatin (P125A). The molecule was produced using standard molecular biology techniques and expressed using mammalian cell culture. The expression vectors of the fusion protein were transiently transfected and expressed in mammalian cells with chemically defined culture media. This protein was purified by Protein A affinity chromatography, dialysed with PBS, concentrated (if needed), and then subjected to 0.2 micron sterile filtration to get the bulk with high purity. The sequence is set out in SEQ ID No. 7, below.

ATF-Linker-hIgG4 Fc-Endostatin (P125A)
MEWSWVFLFFLSVTTGVHSSNELHQVPSNCDCLNGGTCVSNKYFSNIHW
CNCPKKFGGQHCEIDKSKTCYEGNGHFYRGKASTDTMGRPCLPWNSATV
LQQTYHAHRSDALQLGLGKHNYCRNPDNRRRPWCYVQVGLKPLVQECMV
HDCADGSGSGSPPCPPCPAPEFLGGPSVFLFPPKPKDTLMISRTPEVTC
VVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYS
RLTVDKSRWQEGNVFSCSVMHEALHNHYTQKSLSLSLGKGGGGSGGGGS
GGGGSHSHRDFQPVLHLVALNSPLSGGMRGIRGADFQCFQQARAVGLAG
TFRAFLSSRLQDLYSIVRRADRAAVPIVNLKDELLFPSWEALFSGSEGP
LKPGARIFSFDGKDVLRHPTWPQKSVWHGSDANGRRLTESYCETWRTEA
PSATGQASSLLGGRLLGQSAASCHHAYIVLCIENSFMTASK

Purity was 97.6569% as determined by SEC-HPLC. Endotoxin: <1 EU/mg. Concentration was 2 mg/mL. Formulation Buffer was PBS, PH 7.4.

As shown in FIG. 9, SDS-PAGE analysis of the construct in β-mercaptoethanol-reduced (Lane 1) and non-reduced (Lane 2) conditions. Gel stained for 30 minutes with Coomassie Blue. As a result of different reduced proteins migrate as about 63-48 kDa. Non-reduced protein migrates about 135 kDa.

### Example 9—Constructing ATF-Linker-hIgG4 Fc-Endostatin (P125A)

This fusion protein uses ATF bound to a linker, bound to an Fc, then bound to an Endostatin (p125A). The molecule was produced using standard molecular biology techniques and expressed using mammalian cell culture. The expression vectors of the fusion protein were transiently transfected and expressed in mammalian cells with chemically defined culture media. This protein was purified by Protein A affinity chromatography, dialysed with PBS, concentrated (if needed), and then subjected to 0.2 micron sterile filtration to get the bulk with high purity. The sequence is set out in SEQ ID No. 8, below.

ATF-Linker-hIgG4 Fc-Endostatin (P125A)
MEWSWVFLFFLSVTTGVHSSNELHQVPSNCDCLNGGTCVSNKYFSNIHW
CNCPKKFGGQHCEIDKSKTCYEGNGHFYRGKASTDTMGRPCLPWNSATV
LQQTYHAHRSDALQLGLGKHNYCRNPDNRRRPWCYVQVGLKPLVQECMV
HDCADGSGSGSPPCPPCPAPEFLGGPSVFLFPPKPKDTLMISRTPEVTC
VVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYS
RLTVDKSRWQEGNVFSCSVMHEALHNHYTQKSLSLSLGKGGGGSGGGGS
GGGGSHSHRDFQPVLHLVALNSPLSGGMRGIRGADFQCFQQARAVGLAG
TFRAFLSSRLQDLYSIVRRADRAAVPIVNLKDELLFPSWEALFSGSEGP
LKPGARIFSFDGKDVLRHPTWPQKSVWHGSDANGRRLTESYCETWRTEA
PSATGQASSLLGGRLLGQSAASCHHAYIVLCIENSFMTASK

Purity was 99.6569% as determined by SEC-HPLC. Endotoxin: <1 EU/mg. Concentration was 2 mg/mL. Formulation Buffer was PBS, PH 7.4.

As shown in FIG. 10, SDS-PAGE analysis of the construct in β-mercaptoethanol-reduced (Lane 1) and non-reduced (Lane 2) conditions. Gel stained for 30 minutes with Coomassie Blue. As a result of different reduced proteins migrate as about 48-63 kDa. Non-reduced protein migrates about 135 kDa.

### Example 10—Constructing Herceptin-Fc-K5

This fusion protein uses heavy chain Herceptin bound to an Fc and then to K5. The molecule was produced using standard molecular biology techniques and expressed using mammalian cell culture. The expression vectors of the fusion protein were transiently transfected and expressed in mammalian cells with chemically defined culture media. This protein was purified by Protein A affinity chromatography, dialysed with PBS, concentrated (if needed), and then subjected to 0.2 micron sterile filtration to get the bulk with high purity. The sequence is set out in SEQ ID No. 9, below.

Heavy chain (Herceptin)-linker-K5
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGKGGGGSGGGGSGGGGSSEEDCMFGNGKGYRGKRATTVTGTP
CQDWAAQEPHRHSIFTPETNPRAGLEKNYCRNPDGDVGGPWCYTANPRK
LYDYCDVPQCA
Light chain (Herceptin)
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Purity was 99.0715% as determined by SEC-HPLC. Endotoxin: <1 EU/mg. Concentration was 2 mg/mL. Formulation Buffer was PBS, PH 7.4.

As shown in FIG. 11, SDS-PAGE analysis of the construct in β-mercaptoethanol-reduced (Lane 1) and non-reduced (Lane 2) conditions. Gel stained for 30 minutes with Coomassie Blue. As a result of different reduced proteins (Heavy chain and Light chain) migrate as about 63-75 kDa, respectively. Non-reduced protein migrates about and 180-245 kDa.

### Example 11—Constructing ATF-Fc (IgG4)-Linker-Kringle Domains 1-3

This fusion protein uses ATF bound to Fc bound to a linker, bound to Kringle Domains 1-3. The molecule was produced using standard molecular biology techniques and expressed using mammalian cell culture. The expression vectors of the fusion protein were transiently transfected and expressed in mammalian cells with chemically defined culture media. This protein was purified by Protein A affinity chromatography, dialysed with PBS, concentrated (if needed), and then subjected to 0.2 micron sterile filtration to get the bulk with high purity. The sequence is set out in SEQ ID No. 10, below.

ATF-Fc (IgG4)-linker-Kringle Domains 1-3
SNELHQVPSNCDCLNGGTCVSNKYFSNIHWCNCPKKFGGQHCEIDKSKT
CYEGNGHFYRGKASTDTMGRPCLPWNSATVLQQTYHAHRSDALQLGLGK
HNYCRNPDNRRRPWCYVQVGLKPLVQECMVHDCADGSGSGSPPCPPCPA
PEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLP
SSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSV
MHEALHNHYTQKSLSLSLGKGGGGSGGGGSGGGGSSECKTGNGKNYRGT
MSKTKNGITCQKWSSTSPHRPRFSPATHPSEGLEENYCRNPDNDPQGPW
CYTTDPEKRYDYCDILECEEECMHCSGENYDGKISKTMSGLECQAWDSQ
SPHAHGYIPSKFPNKNLKKNYCRNPDRELRPWCFTTDPNKRWELCDIPR
CTTPPPSSGPTYQCLKGTGENYRGNVAVTVSGHTCQHWSAQTPHTHNRT
PENFPCKNLDENYCRNPDGKRAPWCHTTNSQVRWEYCKIPSCDSS

Purity was greater than 90% as determined by SDS-PAGE. Endotoxin: <1 EU/mg. Concentration was 1.3 mg/mL. Formulation Buffer was PBS, PH 7.4.

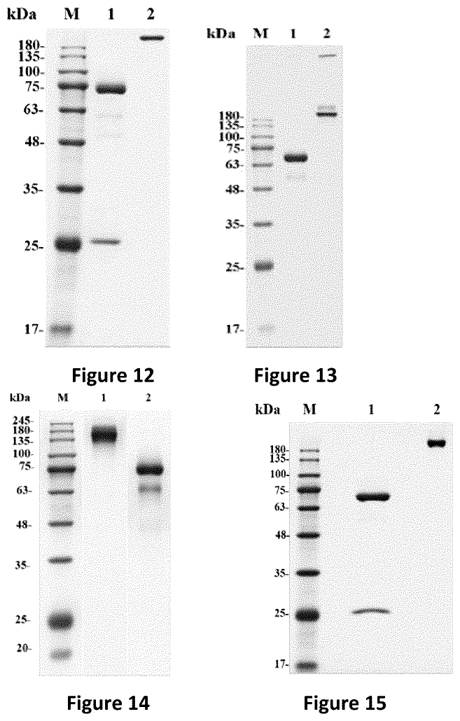

As shown in FIG. 12, SDS-PAGE analysis of the construct in β-mercaptoethanol-reduced (Lane 1) and non-reduced (Lane 2) conditions. Gel stained for 30 minutes with Coomassie Blue. As a result of different reduced proteins migrate as about 63-75 kDa, respectively. Non-reduced protein migrates about and 135-180 kDa.

### Example 12—Constructing Herceptin-Arrestin

This fusion protein uses the heavy chain Herceptin bound to a linker and then to Arrestin. The molecule was produced using standard molecular biology techniques and expressed using mammalian cell culture. The expression vectors of the fusion protein were transiently transfected and expressed in mammalian cells with chemically defined culture media. This protein was purified by Protein A affinity chromatography, dialysed with PBS, concentrated (if needed), and then subjected to 0.2 micron sterile filtration to get the bulk with high purity. The sequence is set out in SEQ ID No. 11, below.

Heavy chain (Herceptin)-linker-Arrestin
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGKGGGGSGGGGSGGGGSSVDHGFLVTRHSQTIDDPQCPSGTK
ILYHGYSLLYVQGNERAHGQDLGTAGSCLRKFSTMPFLFCNINNVCNFA
SRNDYSYWLSTPEPMPMSMAPITGENIRPFISRCAVCEAPAMVMAVHSQ
TIQIPPCPSGWSSLWIGYSFVMHTSAGAEGSGQALASPGSCLEEFRSAP
FIECHGRGTCNYYANAYSFWLATIERSEMFKKPTPSTLKAGELRTHVSR
CQVCMRRT
Light chain (Herceptin)
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Purity was greater than 90% as determined by SDS-PAGE. Endotoxin: <1 EU/mg. Concentration was 1.5 mg/mL. Formulation Buffer was PBS, PH 7.4.

As shown in FIG. 13, SDS-PAGE analysis of the construct in β-mercaptoethanol-reduced (Lane 1) and non-reduced (Lane 2) conditions. Gel stained for 30 minutes with Coomassie Blue. As a result of different reduced proteins (Heavy chain and Light chain) migrate as about 63-75 kDa.

### Example 13—Constructing ATF-huIgG4 Fc-Arrestin

This fusion protein uses ATF bound to an Fc linker and then to Arrestin. The molecule was produced using standard molecular biology techniques and expressed using mammalian cell culture. The expression vectors of the fusion protein were transiently transfected and expressed in mammalian cells with chemically defined culture media. This protein was purified by Protein A affinity chromatography, dialysed with PBS, concentrated (if needed), and then subjected to 0.2 micron sterile filtration to get the bulk with high purity. The sequence is set out in SEQ ID No. 12, below.

ATF-huIgG4 Fc-Arrestin
NELHQVPSNCDCLNGGTCVSNKYFSNIHWCNCPKKFGGQHCEIDKSKTC
YEGNGHFYRGKASTDTMGRPCLPWNSATVLQQTYHAHRSDALQLGLGKH
NYCRNPDNRRRPWCYVQVGLKPLVQECMVHDCADGSGSGSPPCPPCPAP
EFLGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAV
EWESNGQPENNYKTTPPVLDSDGSFFLYSRLTVDKSRWQEGNVFSCSVM
HEALHNHYTQKSLSLSLGKGGGGSGGGGSGGGGSSVDHGFLVTRHSQTI
DDPQCPSGTKILYHGYSLLYVQGNERAHGQDLGTAGSCLRKFSTMPFLF
CNINNVCNFASRNDYSYWLSTPEPMPMSMAPITGENIRPFISRCAVCEA
PAMVMAVHSQTIQIPPCPSGWSSLWIGYSFVMHTSAGAEGSGQALASPG
SCLEEFRSAPFIECHGRGTCNYYANAYSFWLATIERSEMFKKPTPSTLK
AGELRTHVSRCQVCMRRT

Purity was 95.2801% as determined by SEC-HPLC. Endotoxin: <1 EU/mg. Concentration was 2 mg/mL. Formulation Buffer was PBS, PH 7.4.

As shown in FIG. 14, SDS-PAGE analysis of the construct in β-mercaptoethanol-reduced (Lane 1) and non-reduced (Lane 2) conditions. Gel stained for 30 minutes with Coomassie Blue. As a result of different reduced proteins migrate as about 63-75 kDa, respectively. Non-reduced protein migrates about and 180 kDa.

### Example 15—Constructing Anti-PLAUR Recombinant Antibody (Clone ATN-658)—Endostatin P125A

This fusion protein uses the ATN-658 bound to an Endostatin P125A. The molecule was produced using standard molecular biology techniques and expressed using mammalian cell culture. The expression vectors of the fusion protein were transiently transfected and expressed in mammalian cells with chemically defined culture media. This protein was purified by Protein A affinity chromatography, dialysed with PBS, concentrated (if needed), and then subjected to 0.2 micron sterile filtration to get the bulk with high purity. The sequence is set out in SEQ ID No. 13, below.

Heavy Chain Sequence:
EVQLQQSGPELVKTGASVKISCKASGYSFTSYYMHWVKQSHGKSLEWIG
EINPYNGGASYNQKIKGRATFTVDTSSRTAYMQFNSLTSEDSAVYYCAR
SIYGHSVLDYWGQGTSVSVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQK
SLSLSPGKGGGGSGGGGSGGGGSHSHRDFQPVLHLVALNSPLSGGMRGI
RGADFQCFQQARAVGLAGTFRAFLSSRLQDLYSIVRRADRAAVPIVNLK
DELLFPSWEALFSGSEGPLKPGARIFSFDGKDVLRHPTWPQKSVWHGSD
ANGRRLTESYCETWRTEAPSATGQASSLLGGRLLGQSAASCHHAYIVLC
IENSFMTASKDVVMTQTPLTLSVTIGQPASISCKSSQSLLDSDGKTYLN
WLLQRPGQSPKRLIYLVSKLDSGVPDRFTGSGSGTDFTLKISRV
Light Chain Sequence:
EAEDLGVYYCWQGTHFPLTFGAGTKLELKRTVAAPSVFIFPPSDEQLKS
GTASVVCLLNNFYPREAKVQWKVDNALQSGNSQESVTEQDSKDSTYSLS
STLTLSKADYEKHKVYACEVTHQGLSSPVTKSFNRGEC

Purity was 97.26% as determined by SEC-HPLC. Endotoxin: <1 EU/mg. Concentration was 1 mg/mL. Formulation Buffer was PBS, PH 7.4.

As shown in FIG. 15, SDS-PAGE analysis of the construct in β-mercaptoethanol-reduced (Lane 1) and non-reduced (Lane 2) conditions. Gel stained for 30 minutes with Coomassie Blue. Theoretical molecular weights of heavy chain linked with Endostatin P125A mutant protein and light chain are 70.29 kDa and 23.97 kDa, respectively. Here in β-mercaptoethanol-reduced SDS-PAGE, the heavy chain linked with Endostatin P125A mutant protein migrates about 63-75 kDa and light chain migrates about 25 kDa.

### Example 16—in Vivo Anti-Cancer Testing Using Zebrafish Model

The zebrafish model described in Example 2 was used, with the exception that DU145 cells labeled with Dil red fluorescent dye. Approximately 700 Dil-labeled cancer cells were subcutaneously co-implanted into the perivitelline space of 2 days old larvae with Avastin, Avastin-ATF, Herceptin, Herceptin-ATF, Herceptin-K5, and Herceptin-Arrestin at 2 mg/mL, and ATN658, ATN658 K5, ATN658 Endostatin (P125A), ATF-Fc, ATF-Fc-Kringles 1-3, ATF-Fc (IgG4)-Endostatin (P125A), ATF-Fc-K5, and ATF-Fc-Arrestin at 1 mg/mL.

Data are shown as mean±SEM, D'Agostino-Pearson omnibus normality test was performed followed by a Kruskal-Wallis test, two-tailed Student's t-test or Mann-Whitney test where appropriate. Graphs and experimental data were obtained/analysed by using GraphPad Prism v9.0.2.

In summary, results showed that treatment with all the different constructs and Herceptin significantly reduced primary tumor size compared to the negative control after 3 days of treatment. Avastin treatment showed a reduction in the primary tumor size; however, this effect did not reach a mathematical significance. ATF-Fc (IgG4)-Endostatin (P125A) and ATF-Fc-K5 constructs showed the most significant reduction in primary tumor size than the rest of the tested compounds when compared to the negative control.

Results also showed that Avastin and Herceptin constructs had higher anti-tumor efficacy than Avastin and Herceptin treatment alone. Furthermore, ATF-Fc (IgG4)-Endostatin (P125A) and ATF-Fc-K5 constructs had higher anti-tumor efficacy than ATF-Fc alone.

### Study Design

The study was divided into two parts:
Part 1. Test of positive controls
Part 2. Efficacy evaluation of constructs

DU-145 cells were used to evaluate the efficacy of the compounds. The study is set up using 16 groups, divided over 2 days. The DU-145 cells were selected due to a high expression of UPAR, HER2 and VEGF-A, and provide a good base for the positive controls to be able to evaluate the new constructs. The DU-145 cell line also has good engraftment in the zebrafish model, with around 50% of the tumor left after 3 days post-implantation and an average of 10-15 disseminated tumor cells per fish.

### Dose Selection

Cancer cells will be co-injected with the test-compounds. Injection volume is generally 2-5 nL per embryo (usually around 3 nL). To calculate the dosage/embryo, the following formula is used:

3×10\hat{ }−9 L×c(g/L)=ng per embryo

### Avastin

Weekly Avastin treatment in mice (25 mg/kg) was very effective against DU-145 cells (Cancer Chemother Pharmacol (2009) 65:191-195).

In one zebrafish study employing A549 lung cancer cell lines and using Avastin and Endostar (wild type endostatin, not the more active P125A mutant) using bevacizumab at a dose of 28 ng, 83 ng and 250 ng per embryo; endostar at a dose of 11 ng, 33 ng and 99 ng per embryo. In this study, significant inhibition of tumour fluorescence was observed at all doses for both agents although the 33 ng dose of endostar was less efficacious than the flanking doses (Scientific Reports (2018)8:15837). A U-shaped dosing curve has been reported in other studies using endostar (Cancer Gene Therapy (2006) 13:619-627).

In the previous Example 2, 0.25 mg/mL solution of Avastin showed no improvement in tumor volume reduction but did affect angiogenesis against PC3 cells. Thus, the dose in the current experiment will use a higher dose at 0.5 mg/mL with the DU-145 cells prior to implantation, corresponding to 1.5 ng/embryo.

### Herceptin

Near weekly Herceptin treatment in mice (10 mg/kg) showed a moderate (not statistically significant) reduction in tumor volume using DU-145 cells (Cancer Biol. & Therapy (2020) 21:463-475).

Herceptin will be dosed at slightly higher levels (1 mg/mL) than Avastin owing to the reduced sensitivity of DU-145 cells to Herceptin compared to Avastin. This dose translates to 1.8 ng/embryo.

### ATN-658

This unapproved antibody has been studied less intensively than Avastin or Herceptin; however, it has been tested in mice using PC3 transplanted cells and dosed at 10 mg/kg twice a week—yielding significant reductions in tumor volumes and metastatic lesions (Neoplasia (2010) 10:778-88).

When allometrically scaled for zebrafish, this mouse dose would suggest using a 0.3 mg/ml dose (0.9 ng/embryo) when mixed with tumor cells before implantation.

### ATF-Fc Fusions

In Example 2, 0.125 mg/ml of G-Fc-Endostatin (P125A) was both the lowest and best dose for activity using PC3 cells. Thus, this same dose was used for the ATF-Fc fusions (0.375 ng/embryo).

ATF is a combination of an EGF-like domain (G) and a Kringle domain.

All compounds were co-injected with the human tumor cells in zebrafish embryos. Primary tumor size was evaluated directly after implantation and three days post-implantation. Metastasis was evaluated three days post-implantation. The anticancer efficacy will be determined as the change in primary tumor size (i.e., tumor growth or reduction) and the number of tumor cells disseminated to the distal caudal venous plexus (CVP) three days after implantation.

### Part 1. Test of Positive Controls

Before the work plan (Table 2), a dosage test with Avastin and Herceptin was performed to confirm the efficacy of these positive controls (Table 1). In this study, DU-145 cells were tested with the planned dosage, as well as 10 ng/embryo. The higher dosage is selected from previous dosage in zebrafish (Avastin) or mice (Herceptin).

TABLE 1
Dosage testing of Avastin and Herceptin before constructs tests.

| Cell | | | | Group | | Evaluation of | Evaluation of |
|---|---|---|---|---|---|---|---|
| line | Treatment | Conc. | | size | Admin. | primary tumor size | metastasis |
| Du-145 | Vehicle | PBS | | 10 | Co-injected | 0 and 72 h | 72 h |
| Du-145 | Avastin | 0.5 | mg/ml | 10 | Co-injected | 0 and 72 h | 72 h |
| Du-145 | Avastin | 3 | mg/ml | 10 | Co-injected | 0 and 72 h | 72 h |
| Du-145 | Herceptin | 1.0 | mg/ml | 10 | Co-injected | 0 and 72 h | 72 h |
| Du-145 | Herceptin | 3 | mg/ml | 10 | Co-injected | 0 and 72 h | 72 h |

### Part 2. Efficacy Evaluation of Constructs

TABLE 2
Description of experimental groups for efficacy analysis of compounds.

| Cell | | | Group | | Evaluation of | Evaluation of |
|---|---|---|---|---|---|---|
| line | Treatment | Conc. | size | Admin. | primary tumor size | metastasis |
| Du-145 | Avastin | 2 mg/ml | 20 | Co-injected | 0 and 72 h | 72 h |
| Du-145 | Avastin ATF | 2 mg/ml | 20 | Co-injected | 0 and 72 h | 72 h |
| Du-145 | ATN658 | 1 mg/ml | 20 | Co-injected | 0 and 72 h | 72 h |
| Du-145 | ATN658 K5 | 1 mg/ml | 20 | Co-injected | 0 and 72 h | 72 h |
| Du-145 | ATN658 | 1 mg/ml | 20 | Co-injected | 0 and 72 h | 72 h |
| | Endostatin | | | | | |
| | (P125A) | | | | | |
| Du-145 | Herceptin | 2 mg/ml | 20 | Co-injected | 0 and 72 h | 72 h |
| Du-145 | Herceptin ATF | 2 mg/ml | 20 | Co-injected | 0 and 72 h | 72 h |
| Du-145 | Herceptin K5 | 2 mg/ml | 20 | Co-injected | 0 and 72 h | 72 h |
| Du-145 | Herceptin | 2 mg/ml | 20 | Co-injected | 0 and 72 h | 72 h |
| | Arrestin | | | | | |
| Du-145 | ATF-Fc | 1 mg/ml | 20 | Co-injected | 0 and 72 h | 72 h |
| Du-145 | ATF-Fc- | 1 mg/ml | 20 | Co-injected | 0 and 72 h | 72 h |
| | Kringles 1-3 | | | | | |
| Du-145 | ATF-Fe | 1 mg/ml | 20 | Co-injected | 0 and 72 h | 72 h |
| | (IgG4)- | | | | | |
| | Endostatin | | | | | |
| | (P125A) | | | | | |
| Du-145 | ATF-Fc-K5 | 1 mg/ml | 20 | Co-injected | 0 and 72 h | 72 h |
| Du-145 | ATF-Fc- | 1 mg/ml | 20 | Co-injected | 0 and 72 h | 72 h |
| | Arrestin | | | | | |
| Du-145 | Vehicle | PBS | 20 | | 0 and 72 h | 72 h |

### Results

### Part 1. Test of Positive Controls

Before the efficacy study, a dosage test with Avastin and Herceptin was performed to confirm the efficacy of these positive controls and to decide on dosage for the efficacy study.

Results showed that treatment with Herceptin exhibited a borderline significant reduction of primary tumor size compared to the negative control at 3 mg/ml. In contrast, treatment with Avastin showed no effect on primary tumor size compared to the negative control (FIG. 16).

FIG. 16 also shows that the number of disseminated cancer cells was reduced in the presence of Avastin at 3 mg/ml and Herceptin at 1 mg/mL compared to the negative control after three days of treatment; however, this effect did not reach a mathematical significance. FIG. 16 is an evaluation of anti-tumor and anti-metastatic efficacy of Avastin and Herceptin on DU145 cancer cells after 72 hours of exposure. Cancer cells were subcutaneously co-implanted±Avastin±Herceptin into the perivitelline space of 2 days old zebrafish embryos as described in the study plan. Data are presented as mean±SEM, two-tailed Student's t-test was performed.

### Part 2. Efficacy Evaluation of Constructs

After part 1, the concentration of positive controls and constructs to test in the efficacy evaluation was decided (see Table 2).

### Primary Tumor

Results are first presented as a comparison of the primary tumor size between the different compounds by grouping them according to the fusion construct, i.e., ATF constructs, ATN constructs, Avastin and Herceptin constructs, and K5, Arrestin, and P125 constructs. Various constructs can be found in multiple graphs. All constructs were compiled and presented in a single graph where anti-tumor efficacy is compared to the negative control.

### ATF Constructs

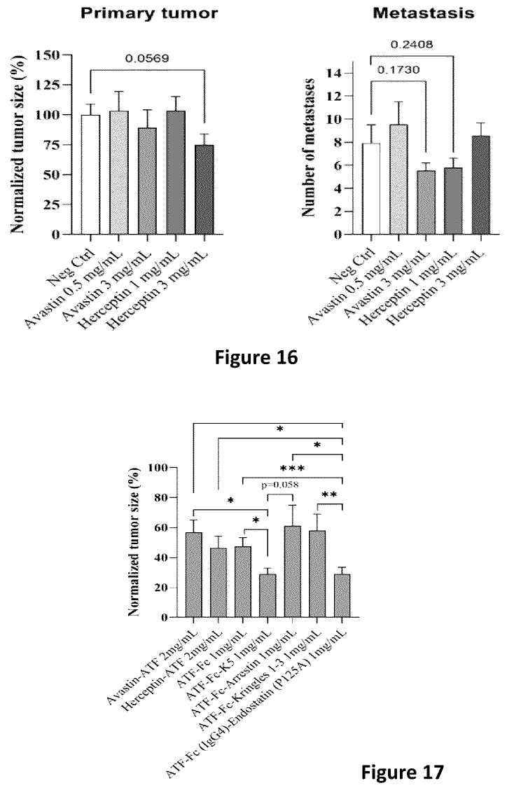

Results showed that ATF-Fc-K5 and ATF-Fc (IgG4)-Endostatin (P125A) constructs showed a significant reduction of primary tumor size compared to the rest of the ATF constructs. Meanwhile, Avastin ATF, Herceptin ATF, ATF-Fc, ATF-Fc-Arrestin, and ATF-Fc-Kringles 1-3 showed similar, non-significantly different, primary tumor size after three days of treatment (FIG. 17).

FIG. 17 is a comparison of DU145 primary tumor size between the different ATF constructs after 72 hours of exposure. Data are presented as mean±SEM, D'Agostino-Pearson omnibus normality test was performed. Kruskal-Wallis test was performed (p<0.0001) followed by a two-tailed Student's t-test or Mann-Whitney test where appropriate. *p<0.05, **p<0.01, and ***p<0.001.

### Avastin and Herceptin Constructs

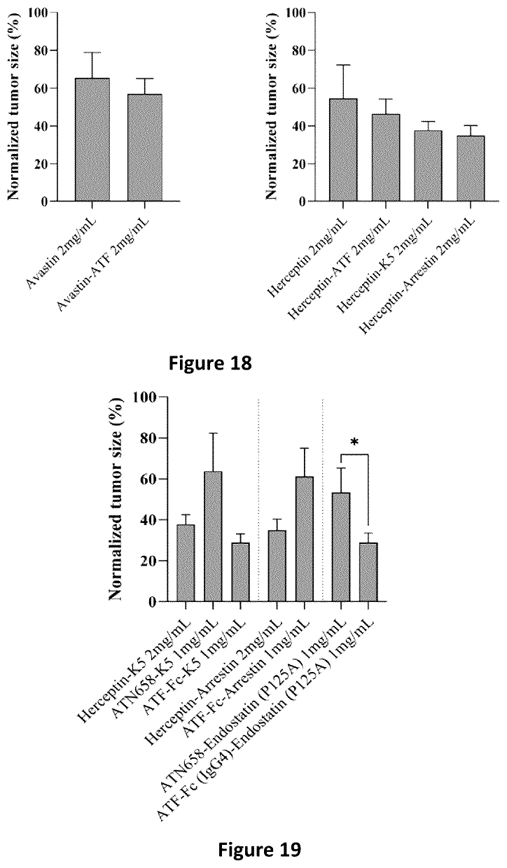

FIG. 18 is a comparison of DU145 primary tumor size between the different Avastin and Herceptin constructs after 72 hours of exposure. Data are presented as mean±SEM.

### K5, Arrestin, and P125A Constructs

Primary tumor size when comparing K5 or Arrestin constructs treatment after 3 days is shown in FIG. 19. The ATF-Fc (IgG4)-Endostatin (P125A) showed a significantly reduced primary tumor size compared to the ATN658 Endostatin (P125A) construct treatment after 3 days (FIG. 19). Data are presented as mean±SEM, D'Agostino-Pearson omnibus normality test was performed. Kruskal-Wallis test was performed (p<0.0001) followed by a two-tailed Student's t-test or Mann-Whitney test where appropriate. *p<0.05.

### All Constructs

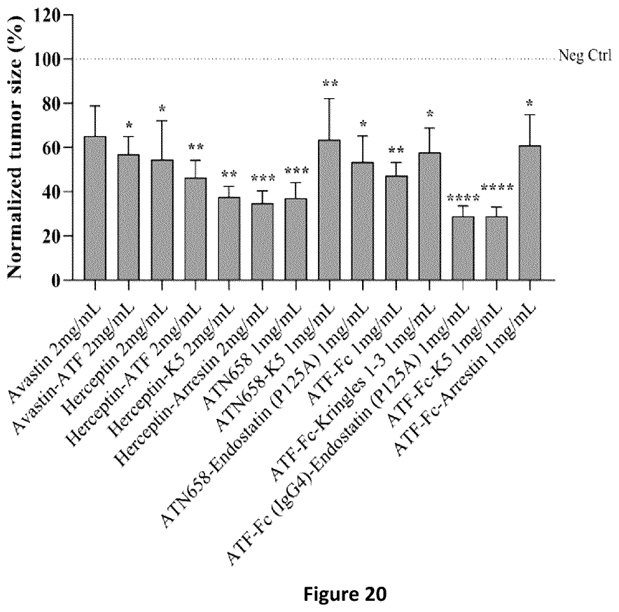

Results showed that treatment with all the different constructs and Herceptin significantly reduced primary tumor size compared to the negative control after 3 days of treatment (FIG. 20). Meanwhile, Avastin treatment showed a reduction in the primary tumor size; however, this effect did not reach a mathematical significance (FIG. 20).

FIG. 20 shows that ATF-Fc (IgG4)-Endostatin (P125A) and ATF-Fc-K5 constructs showed the most significant reduction in primary tumor size compared to the rest of the tested compounds.

FIG. 20 also shows that Herceptin-Arrestin construct exhibited a higher anti-tumor efficacy than Herceptin, Herceptin-ATF, and Herceptin-K5 constructs. Data are presented as mean±SEM, D'Agostino-Pearson omnibus normality test was performed. Kruskal-Wallis test was performed (p<0.0001) followed by a two-tailed Student's t-test or Mann-Whitney test where appropriate. *p<0.05, **p<0.01, ***p<0.001, and ****p<0.0001.

### Metastasis

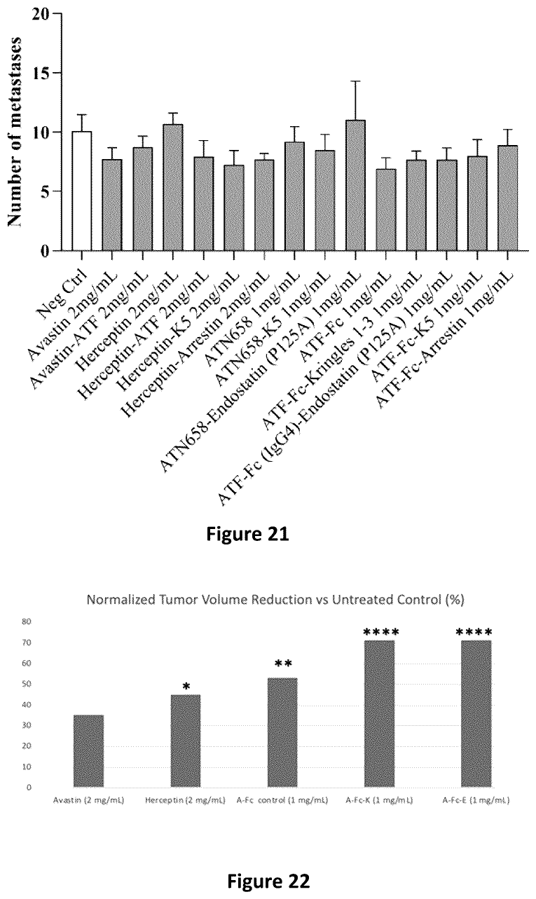

Results showed that a decreased metastatic dissemination of DU145 cells was observed in the presence of Avastin, Herceptin-K5, Herceptin-Arrestin, ATF-Fc, and ATF-Fc-Kringles 1-3 treatments after three days compared to the negative control. However, this effect did not reach a mathematical significance (FIG. 21).

### Conclusions

Avastin and Herceptin constructs have higher anti-tumor efficacy than Avastin and Herceptin treatment alone. Furthermore, ATF-Fc (IgG4)-Endostatin (P125A) and ATF-Fc-K5 constructs have higher anti-tumor efficacy than ATF-Fc alone.

All of the evaluated treatments reduce the dissemination of DU145.

### Example 17—in Vivo Anti-Cancer Testing Using Zebrafish Model

The zebrafish model described in Example 16 was used.

FIG. 22 shows that the ATF-Fc-K5 and ATF-Fc-Endostatin (P125A) constructs show an improved normalized tumor volume reduction versus untreated control. Data are presented as mean±SEM, D'Agostino-Pearson omnibus normality test was performed. Kruskal-Wallis test was performed (p<0.0001) followed by a two-tailed Student's t-test or Mann-Whitney test where appropriate. *p<0.05, **p<0.01, ***p<0.001, and ****p<0.0001.

Although the present disclosure is described, at least in part, in terms of methods, a person of ordinary skill in the art will understand that the present disclosure is also directed to the various components for performing at least some of the aspects and features of the described methods. The present disclosure may be embodied in other specific forms without departing from the subject matter of the claims. The described example embodiments are to be considered in all respects as being only illustrative and not restrictive. Selected features from one or more of the above-described embodiments may be combined to create alternative embodiments not explicitly described, features suitable for such combinations being understood within the scope of this disclosure.

1. A compound comprising:
a first binding peptide from a urokinase-type plasminogen receptor (uPAR) antagonist having activity of a uPAR antangonist, operatively linked to a N-terminal of an immunoglobulin G (IgG) antibody peptide operatively linked to
a C-terminal of the immunoglobulin G (IgG) antibody peptide operatively linked to a second binding peptide from an endostatin derived peptide sequence having endostatin activity or plasminogen derived peptide sequence having plasminogen activity.

2. The compound of claim 1, wherein the IgG is a fragment of an Fc fusion peptide.

3. The compound of claim 1, wherein the IgG is a fragment of a human Fc fusion peptide.

4. (canceled)

5. The compound of claim 2, wherein the Fc fusion protein is selected from the group consisting of an IgG1 isotype Fc peptide, an IgG2 isotype Fc peptide, an IgG3 isotype Fc peptide, an IgG4 isotype Fc peptide and an IgG protein.

6. The compound of claim 1, wherein the first binding peptide comprises an N terminal fragment of uPAR having uPAR activity.

7. The compound of claim 1, wherein the first binding peptide comprises a heavy chain of the first binding peptide only.

8. The compound of claim 1, wherein the first binding peptide is selected from the group consisting of EGF-like domain G, an N-terminal portion of the ATF from a urokinase-type plasminogen receptor (uPAR) antagonist, antibody 2G10, antibody ATN-658, antibody 8B12.

9. The compound of claim 1, wherein the first binding peptide comprises a fragment of heavy and light sequences of antibodies that block uPAR signaling.

10. The compound of claim 1, wherein the second binding peptide is selected from the group consisting of a fragment of an endostatin having endostatin activity, an endostatin, a fragment of a plasminogen having plasminogen activity, an endostatin with a P125mutation, a kringle domain of plasminogen, kringle domains 1 to 5 of plasminogen, and kringle domain 5 from a plasminogen.

11. (canceled)

12. The compound of claim 1, further comprising a linker peptide between the antibody peptide and the first binding peptide.

13. The compound of claim 1, further comprising a linker peptide between the antibody peptide and the second binding peptide.

14. The compound of claim 1, wherein the compound comprises a peptide coded by a sequence selected from the group consisting of SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, and SEQ ID No. 13.

15. (canceled)

16. (canceled)

17. A compound comprising a compound at least 70%, at least 75%, at least 80%, at least 85%, at least 90%, at least 95%, or at least 99% identical to the peptide coded by a sequence selected from the group consisting of SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, and SEQ ID No. 13.

18. A compound selected from the group consisting of G-Fc-Endostatin (P125A), ATN658-Fc-K5, Herceptin-Fc-ATF, Avastin-Fc-ATF, Human IgG1 Fc-linker-ATF, ATF-Linker-hIgG4 Fc-Kringle domain K5, ATF-Linker-hIgG4 Fc-Endostatin (P125A), ATF-Linker-hIgG4 Fc-Endostatin (P125A), Herceptin-Fc-K5, ATF-Fc (IgG4)-linker-Kringle Domains 1-3, Herceptin-Arrestin, ATF-huIgG4 Fc-Arrestin and ATN658-Endostatin (P125).

19. (canceled)

20. A nucleotide encoding a compound of claim 1.

21. The compound of claim 1, wherein the compound exhibits anti-cancer activity.

22. A method of treating cancer comprising administration of a composition comprising the compound of claim 1.

23. (canceled)

24. A vector containing a compound of claim 1.

25. The method or use of claim 22 wherein the cancer is a solid cancer.

26. A method for inhibiting metastasis in a subject with cancer, the method comprising administering an effective amount of the compound of claim 1 to the subject.

27. (canceled)
